(19) United States
(12) Patent Application Publication
(10) Pub. No.: US 2009/0149505 A1
(43) Pub. Date: Jun. 11, 2009

(54) Metabotropic Glutamate Receptor-Potentiating Isoindolones

(75) Inventors: James R. Empfield, Wilmington, DE (US); James J. Folmer, Wilmington, DE (US); James R. Arnold, San Francisco, CA (US); Joshua Clayton, Oakville (CA); Abdelmalik Slassi, Mississauga (CA); Methvin Isaac, Brampton (CA); Ian Egle, Georgetown (CA); Fupeng Ma, Melrose, MA (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/279,124

(22) PCT Filed: Feb. 6, 2007
(86) PCT No.: PCT/US07/03233

§ 371 Date: Oct. 29, 2008

Related U.S. Application Data
(60) Provisional application No. 60/773,870, filed on Feb. 16, 2006.
(60) Provisional application No. 60/871,590, filed on Dec. 22, 2006.

(51) Int. Cl. C07D 401/10; A61K 31/4439; A61K 31/4035; C07D 209/44; A61P 9/00; A61P 25/00; A61P 25/28; A61P 29/00; A61P 25/08

(52) U.S. Cl. 514/339; 546/277.1; 514/416; 548/472; 546/275.4

(57) ABSTRACT

Compounds of Formula I:

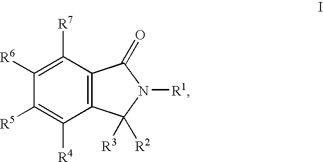

wherein R¹, R², R³, R⁴, R⁵, R⁶ and R⁷ are as defined in the description, processes for the preparing such compounds, new intermediates employed in their preparation, pharmaceutical compositions containing the compounds, and uses of the compounds in therapy.

### CROSS REFERENCE TO RELATED APPLICATIONS

None.

### BACKGROUND OF THE INVENTION

This invention relates to potentiators of glutamate receptors, methods for their preparation, pharmaceutical compositions containing them and their use in therapy.

The metabotropic glutamate receptors (mGluR) are a family of GTP-binding-protein (G-protein) coupled receptors that are activated by glutamate, and that have important roles in synaptic activity in the central nervous system, neural plasticity, neural development and neurodegeneration.

Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels (Schoepp et al., 1993, Trends Pharmacol. Sci., 14:13; Schoepp, 1994, Neurochem. Int., 24:439; Pin et al., 1995, Neuropharmacology 34:1; Bordi & Ugolini, 1999, Prog. Neurobiol. 59:55).

Eight mGluR subtypes have been identified. The subtypes are divided into three groups based upon primary sequence similarity, signal transduction linkages, and pharmacological profile. Group-I includes mGluR1 and mGluR5, which activate phospholipase C and the generation of an intracellular calcium signal. Group-II (mGluR2 and mGluR3) and Group-III (mGluR4, mGluR6, mGluR7, and mGluR8) mGluRs mediate an inhibition of adenylyl cyclase activity and cyclic AMP levels. For a review, see Pin et al., 1999, Eur. J. Pharmacol., 375:277-294.

Activity of mGluR family receptors is implicated in a number of normal processes in the mammalian CNS, and are important targets for compounds for the treatment of a variety of neurological and psychiatric disorders. Activation of mGluRs is required for induction of hippocampal long-term potentiation and cerebellar long-term depression (Bashir et al., 1993, Nature, 363:347; Bortolotto et al., 1994, Nature, 368:740; Aiba et al., 1994, Cell, 79:365 Aiba et al., 1994, Cell, 79:377). A role for mGluR activation in nociception and analgesia also has been demonstrated (Meller et al., 1993, Neuroreport, 4: 879; Bordi & Ugolini, 1999, Brain Res., 871:223). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex (Nakanishi, 1994, Neuron, 13:1031; Pin et al., 1995, Neuropharmacology, see above; Knopfel et al., 1995, J. Med. Chem., 38:1417).

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

### SUMMARY OF THE INVENTION

We have identified a class of compounds that modulate mGluR function. Such compounds are compounds of Formula I,

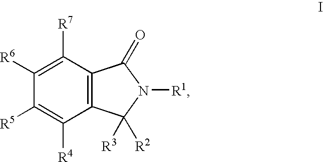

wherein:

R¹ is —CHR⁸R⁹;

R², R³ and R⁴ are H;

R⁶ and R⁷ are independently selected from the group consisting of H, halogen, C₁₋₆-alkyl and C₀₋₆-alkylaryl;

R⁵ is selected from the group consisting of C₁₋₆-alkyl, C₀₋₆-alkylaryl, C₀₋₆-alkylheteroaryl and C₀₋₆-alkylheterocyclyl; wherein, when chemically-feasible, R⁵ may be substituted by one or more A, and wherein any cyclic moiety is optionally fused to a 5- to 7-membered ring that may have one or more heteroatoms independently selected from the group consisting of N, O and S;

A is selected from the group consisting of C₁₋₆-alkyl, C₀₋₆-alkylaryl, C₀₋₆-alkylheteroaryl, C₀₋₆-alkylheterocyclyl, C₀₋₆-alkyl(CO)N(R¹⁰)₂, C₀₋₆-alkylNR¹⁰(CO)R¹⁰, C₀₋₆-alkyl(SO₂)N(R¹⁰)₂, C₀₋₆-alkylNR¹⁰(SO₂)R¹⁰ and a 5- to 7-membered ring that may have one or more heteroatoms independently selected from the group consisting of N, O and S, wherein said 5- to 7-membered ring is optionally substituted by one or more R¹⁰;

R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;

n is 1, 2, 3, 4, 5 or 6;

X is S or O, and

R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy.

In another aspect we describe pharmaceutically-acceptable salts, hydrates, solvates, optical isomers, or combination thereof of compounds of Formula I; processes for the preparation of such compounds; pharmaceutical compositions comprising such compounds according to Formula I together with a pharmaceutically-acceptable carrier or excipient; methods for the treatment or prevention of neurological and psychiatric disorders associated with glutamate dysfunction in an animal in need of such treatment comprising a step of administering to an animal a therapeutically effective amount of a compound of Formula I or a pharmaceutical composition thereof.

In a further aspect we describe the use of a compound according to Formula I, or a pharmaceutically acceptable salt or solvate thereof, in the manufacture of a medicament for the treatment of any of the conditions discussed herein and, further, provide compounds of Formula I, or pharmaceutically acceptable salts or solvates thereof, for use in therapy.

### DETAILED DESCRIPTION OF THE INVENTION

The present invention is based upon the discovery that activity of metabotropic glutamate receptors can be modulated by certain compounds. Particularly, it has been discovered that described compounds potentiate the activity of the mGluR2 receptor. Such compounds are compounds in accord with Formula I that are useful in therapy, in particular as pharmaceuticals for the treatment of neurological and psychiatric disorders associated with glutamate dysfunction.

### Compounds:

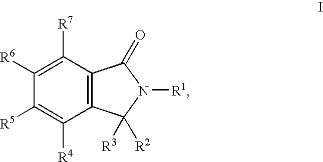

wherein:

R¹ is —CHR⁸R⁹;

R², R³ and R⁴ are H;

R⁶ and R⁷ are independently selected from the group consisting of H, halogen, C₁₋₆-alkyl and C₀₋₆-alkylaryl;

R⁵ is selected from the group consisting of C₁₋₆-alkyl, C₀₋₆-alkylaryl, C₀₋₆-alkylheteroaryl and C₀₋₆-alkylheterocyclyl; wherein, when chemically-feasible, R⁵ may be substituted by one or more A, and wherein any cyclic moiety is optionally fused to a 5- to 7-membered ring that may have one or more heteroatoms independently selected from the group consisting of N, O and S;

A is selected from the group consisting of C₁₋₆-alkyl, C₀₋₆-alkylaryl, C₀₋₆-alkylheteroaryl, C₀₋₆-alkylheterocyclyl, C₀₋₆-alkyl(CO)N(R¹⁰)₂, C₀₋₆-alkylNR¹⁰(CO)R¹⁰, C₀₋₆-alkyl(SO₂)N(R¹⁰)₂, C₀₋₆-alkylNR¹⁰(SO₂)R¹⁰ and a 5- to 7-membered ring that may have one or more heteroatoms independently selected from the group consisting of N, O and S, wherein said 5- to 7-membered ring is optionally substituted by one or more R¹⁰;

R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;

n is 1, 2, 3, 4, 5 or 6;

X is S or O, and

R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;

or a pharmaceutically acceptable salt, hydrate, solvate, optical isomer, or combination thereof.

Particular compounds are those in accord with Formula I wherein:

R¹ is —CHR⁸R⁹;

R², R³ and R⁴ are H;

R⁶ is selected from the group consisting of H, halogen and C₁₋₆-alkyl;

R⁷ is selected from the group consisting of halogen and C₁₋₆-alkyl;

R⁵ is a 5- to 7-membered ring that may have one or more heteroatoms independently selected from the group consisting of N, O and S, wherein, when chemically-feasible, R⁵ may be substituted by one or more A;

A is selected from the group consisting of C₁₋₆-alkyl, C₀₋₆-alkylaryl, C₀₋₆-alkylheteroaryl, C₀₋₆-alkylheterocyclyl, C₀₋₆-alkyl(CO)N(R¹⁰)₂, C₀₋₆-alkylNR¹⁰(CO)R¹⁰, C₀₋₆-alkyl(SO₂)N(R¹⁰)₂, C₀₋₆-alkylNR¹⁰(SO₂)R¹⁰ and a 5- to 7-membered ring that may have one or more heteroatoms independently selected from the group consisting of N, O and S, wherein said 5- to 7-membered ring is optionally substituted by one or more R¹⁰;

R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;

n is 1, 2 or 3;

X is S or O;

R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;

or a pharmaceutically acceptable salt, hydrate, solvate, optical isomer, or combination thereof.

Other particular compounds according to Formula I, are those wherein:

R¹ is —CHR⁸R⁹;

R², R³ and R⁴ are H;

R⁶ is selected from the group consisting of H, halogen and C₁₋₆-alkyl;

R⁷ is selected from the group consisting of halogen and C₁₋₆-alkyl;

R⁵ is a 5- to 7-membered ring that may have one or more heteroatoms independently selected from the group consisting of N, O and S, wherein, where chemically-feasible, R⁵ may be substituted by one or more A, and wherein any cyclic moiety is optionally fused to a 5- to 7-membered ring that may have one or more heteroatoms independently selected from the group consisting of N, O and S;

A is C₀₋₆-alkyl(CO)N(R¹⁰)₂, C₀₋₆-alkylNR¹⁰(CO)R¹⁰, C₀₋₆-alkyl(SO₂)N(R¹⁰), or C₀₋₆-alkylNR¹⁰(SO₂)R¹⁰;

R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;

n is 1, 2 or 3;

X is S or O, and

R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;

or a pharmaceutically acceptable salt, hydrate, solvate, optical isomer, or combination thereof.

Yet other particular compounds according to Formula I, are those wherein:

R¹ is —CHR⁸R⁹;

R², R³ and R⁴ are H;

R⁶ is selected from the group consisting of H, halogen and C₁₋₆-alkyl;

R⁷ is selected from the group consisting of halogen and C₁₋₆-alkyl;

R⁵ is phenyl or pyridyl;

A is C₀₋₆-alkyl(CO)N(R¹⁰)₂, C₀₋₆-alkylNR¹⁰(CO)R¹⁰, C₀₋₆-alkyl(SO₂)N(R¹⁰)₂ or C₀₋₆-alkylNR¹⁰(SO₂)R¹⁰;

R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;

n is 1, 2 or 3;

X is S or O, and

R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;

or a pharmaceutically acceptable salt, hydrate, solvate, optical isomer, or combination thereof.

Still other particular compounds in accord with Formula I are those mentioned in the Examples herein.

It will be understood by those of skill in the art that when compounds of Formula I have one or more chiral centers, such compounds may exist in, and be isolated as, enantiomeric or diastereomeric forms, or as a racemic mixture and are encompassed within the scope of the description. Such optically active forms of compounds may be prepared, for example, by chiral chromatographic separation of a racemate, by synthesis from optically active starting materials or by asymmetric synthesis based on the procedures described thereafter.

It will also be appreciated by those of skill in the art that certain compounds of Formula I may exist as geometrical isomers, for example E and Z isomers of alkenes which are encompassed by the scope of the description. It will further be understood that certain compounds of Formula I may exist as tautomers.

It will also be understood by those of skill in the art that certain compounds of Formula I may exist in solvated, for example hydrated, as well as unsolvated forms and that all such solvated forms of the compounds of Formula I are within the scope of the description.

Salts of compounds of Formula I are within the scope of the description. Generally, pharmaceutically acceptable salts of compounds of Formula I are obtained using standard procedures well known in the art, for example, by reacting a sufficiently basic compound, for example an alkyl amine with a suitable acid, for example, HCl or acetic acid, to afford a physiologically acceptable anion. It is also possible to make a corresponding alkali metal (such as sodium, potassium, or lithium) or an alkaline earth metal (such as a calcium) salt by treating a compound of Formula I having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment a compound of Formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate. Other embodiments include the compounds described herein, their pharmaceutically acceptable salts, hydrates, solvates and optical isomers thereof.

### Pharmaceutical Compositions:

Compounds of Formula I may be formulated into conventional pharmaceutical composition comprising such a compound, or a pharmaceutically acceptable salt or solvate thereof, in association with a pharmaceutically acceptable carrier or excipient. Such pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or table disintegrating agents. A solid carrier can also be an encapsulating material.

In powders, the carrier is a finely divided solid, which is in admixture with a finely divided compound, the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the active compound, all percentages by weight being based on the total weight of the composition.

A therapeutically effective amount of a compound of Formula I can be determined by one of ordinary skill in the art using known criteria including the age, weight and response of the individual patient, and interpreted within the context of the disease which is being treated or which is being prevented.

### Medical Use:

We have observed that the described compounds act as modulators of metabotropic glutamate receptors and contemplate that such compounds will exhibit activity as pharmaceuticals. More particularly, the described compounds exhibit activity as potentiators of the mGluR2 receptor, and will be useful in therapy, in particular for the treatment of neurological and psychiatric disorders associated with glutamate dysfunction in an animal.

More specifically, the neurological and psychiatric disorders include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's disease, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD)), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), attention deficit/hyperactivity disorder, and conduct disorder.

We thus provides a use of any of the compounds according to Formula I, or a pharmaceutically acceptable salt or solvate thereof, for the manufacture of a medicament for the treatment of any of the conditions discussed above.

Compounds of Formula I or pharmaceutically acceptable salts, solvates or in vivo hydrolysable esters thereof, or pharmaceutical compositions or formulations comprising a compound of Formula I may be administered concurrently, simultaneously, sequentially or separately with another pharmaceutically active compound or compounds selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof
(ii) atypical antipsychotics including for example quetiapine and pharmaceutically active isomer(s) and metabolite(s) thereof amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, risperidone, quetiapine, sertindole, sulpiride, suproclone, suriclone, thioridazine, trifluoperazine, trimetozine, valproate, valproic acid, zopiclone, zotepine, ziprasidone and equivalents thereof.
(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof.
(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazeparn, quazepam, reclazepam, tracazolate, trepipam, temazepam, triazolam, uldazepam, zolazepam and equivalents and pharmaceutically active isomer(s) and metabolite(s) thereof.
(v) anticonvulsants including for example carbamazepine, valproate, lamotrogine, gabapentin and equivalents and pharmaceutically active isomer(s) and metabolite(s) thereof.
(vi) Alzheimer's therapies including for example donepezil, memantine, tacrine and equivalents and pharmaceutically active isomer(s) and metabolite(s) thereof.
(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.
(viii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof.
(ix) stroke therapies including for example abciximab, activase, NXY-059, citicoline, crobenetine, desmoteplase, repinotan, traxoprodil and equivalents and pharmaceutically active isomer(s) and metabolite(s) thereof.
(x) over active bladder urinary incontinence therapies including for example darafenacin, falvoxate, oxybutynin, propiverine, robalzotan, solifenacin, tolterodine and equivalents and pharmaceutically active isomer(s) and metabolite(s) thereof.
(xi) neuropathic pain therapies including for example gabapentin, lidoderm, pregablin and equivalents and pharmaceutically active isomer(s) and metabolite(s) thereof.
(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof.
(xiii) insomnia therapies including for example allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents and pharmaceutically active isomer(s) and metabolite(s) thereof.
(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.
Such combination products employ the compounds of this invention within the dosage range described herein and the other pharmaceutically active compound or compounds within approved dosage ranges and/or the dosage described in the publication reference.

Additionally, we provide a method for the treatment of a subject suffering from any of the conditions discussed herein, whereby an effective amount of a compound according to Formula I or a pharmaceutically acceptable salt or solvate thereof, is administered to a patient in need of such treatment. Thus we provide compounds of Formula I or pharmaceutically acceptable salts or solvates thereof, as hereinbefore defined for use in therapy.

In the context of the present specification, the term “therapy” also includes “prophylaxis” unless there are specific indications to the contrary. The term “therapeutic” and “therapeutically” should be construed accordingly. The term “therapy” within the present context further encompasses the administration of an effective amount of a compound of Formula I, to mitigate either a pre-existing disease state, acute or chronic, or to mitigate a recurring condition. This definition also encompasses prophylactic therapies for prevention of recurring conditions and continued therapy for chronic disorders.

In use for therapy in a warm-blooded animal such as a human, compounds of Formula I may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints. In particular embodiments the route of administration will be oral, intravenous, or intramuscular.

The dosage will depend on the route of administration, the severity of the disease, age and weight of the patient and other factors normally considered by the attending physician, who determines the individual regimen and dosage level for a particular patient.

As mentioned above, compounds of Formula I may be provided or delivered in a form suitable for oral use, for example, in a tablet, lozenge, hard and soft capsule, aqueous solution, oily solution, emulsion, and suspension. Alternatively, such compounds may be formulated into a topical administration, for example, as a cream, ointment, gel, spray, or aqueous solution, oily solution, emulsion or suspension. Compounds of Formula I also may be provided in a form that is suitable for nasal administration, for example, as a nasal spray, nasal drops, or dry powder. The compounds can be administered to the vagina or rectum in the form of a suppository. Compounds of Formula I may also be administered parentally, for example, by intravenous, intravesicular, subcutaneous, or intramuscular injection or infusion. The compounds can be administered by insufflation (for example as a finely divided powder). The compounds may also be administered transdermally or sublingually.

In addition to their use in therapeutic medicine, the compounds of Formula I, or salts thereof, are useful as pharmacological tools in the development and standardization of in vitro and in vivo test systems for the evaluation of the effects of inhibitors of mGluR-related activity in laboratory animals as part of the search for new therapeutics agents. Such animals include, for example, cats, dogs, rabbits, monkeys, rats and mice.

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “alkyl” as used herein means a straight- or branched-chain hydrocarbon radical having for example from one to six carbon atoms, and includes methyl, ethyl, propyl, isopropyl, t-butyl and the like.

The term “alkoxy” as used herein means a straight- or branched-chain alkoxy radical having for example from one to six carbon atoms and includes methoxy, ethoxy, propyloxy, isopropyloxy, t-butoxy and the like.

The term “halo” as used herein means halogen and includes fluoro, chloro, bromo, iodo and the like, in both radioactive and non-radioactive forms.

The term “haloalkyl” as used herein means an alkyl group in which at least one H atom has been replaced by a halo atom, and includes groups such as CF₃, CH₂Br and the like.

The term “alkylene” as used herein means a difunctional branched or unbranched saturated hydrocarbon radical having for example one to six carbon atoms, and includes methylene, ethylene, n-propylene, n-butylene and the like.

The term “aryl” as used herein means an aromatic group having five to twelve atoms, and includes phenyl, naphthyl and the like.

The term “heteroaryl” means an aromatic group which includes at least one heteroatom selected from the group consisting of N, S and O, and includes groups such as pyridyl, indolyl, furyl, benzofuryl, thienyl, benzothienyl, quinolyl, oxazolyl and the like.

The term “heterocyclyl” means a saturated or partially-saturated group which includes at least one heteroatom selected from the group consisting of N, S and O, and includes groups such as morpholinyl, piperidinyl, piperazinyl, pyrrolidinyl and the like.

The term “5- to 7-membered ring” includes aryl rings, heterocyclyl rings or heteroaryl rings.

ACN means acetonitrile; RT means room temperature; DME means dimethoxyethane; DMSO means dimethylsulfoxide; EtOAc means ethyl acetate; TFA means trifluoroacetic acid; EtOH means ethanol, and GMF means glass microfiber.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

“Solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents are ethanol, water and the like. When water is the solvent, the molecule is referred to as a hydrate.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound of Formula I which is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form suitable for administration to a patient. One example of such a carrier is a pharmaceutically acceptable oil typically used for parenteral administration.

### Exemplary Methods:

### Purification

### Method A: Normal Phase Chromatography

Flash chromatography employed as a method for purification for selected compounds and intermediates. Isco CombiFlash Sq 16x or Companion instrument: pre-packaged disposable RediSep SiO₂ stationary phase columns (4, 12, 40, 120 gram sizes) with gradient elution at 5-125 mL/min of selected bi-solvent mixture, UV detection (190-760 nm range) or timed collection, 0.1 mm flow cell path length.

### Method B: Preparative Reverse-Phase HPLC

Reverse Phase High Pressure Liquid Chromatography (RP-HPLC) was employed as a method of purification for selected compounds. Gilson instrumentation (215 Injector, 333 Pumps and 155 UV/Vis Detector): Varian C8 reverse phase column (60 Angstrom irregular load in 8 μm particle size, 21 mm ID×25 cm). The compounds were solubilized in dimethyl sulfoxide: methanol (˜1:1). Gradient elution performed with aqueous 0.1% trifluoroacetic acid/ACN (typically 25-75% ACN over 30 minutes, 95% ACN over 7 minutes) flow rate at 22 mL/min, UV collection at 254 nm. Retention time (t_{R})=mins.

### Microwave Heating Instrumentation:

Personal Chemistry Smith Synthesizer or Optimizer microwave units (monomodal, 2.45 GHz, 300 W max) were utilized for microwave heating of reactions.

### LC-MS HPLC Conditions:

Column: Agilent Zorbax SB-C8, 5 μm, 2.1 mm ID×50 mm Flow: 1.4 mL/min
Gradient: 95% A to 90% B over 3 minutes hold 1 minute ramp down to 95% A over 1 minute and hold 1 minute. Where A=2% ACN in water with 0.1% formic acid and B=2% water in ACN with 0.1% formic acid. UV-DAD 210-400 nm.

### Processes for Preparing:

The selection of a particular method to prepare a given compound is within the purview of the person of skill in the art. The choice of particular structural features and/or substituents may therefore influence the selection of one method over another. Within these general guidelines, methods described herein can be used to prepare compounds of Formula I. Unless indicated otherwise, the variables described in the following schemes and methods have the same definitions as those given for Formula I herein.

### Synthetic Methods:

### EXAMPLE 1

### 2-Cyclopropyl-7-methyl-5-pyridin-3-yl-2,3-dihydro-isoindol-1-one

2-Cyclopropyl-7-methyl-5-pyridin-3-yl-2,3-dihydro-isoindol-1-one (B) was prepared as illustrated in Scheme 1.

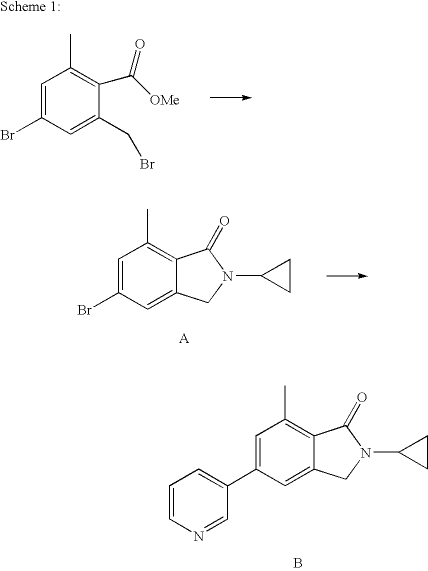

### a) 5-Bromo-2-cyclopropyl-7-methyl-2,3-dihydro-isoindol-1-one

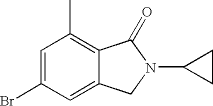

To a stirred solution of 4-bromo-2-bromomethyl-6-methyl-benzoic acid methyl ester (400 mg, 1.24 mmol) in 5 mL of ACN was added cyclopropylamine (356 μL, 3.74 mmol) K₂CO₃ (515 mg, 3.74 mmol) and boronic acid (15 mg, 0.248 mmol). The reaction was heated to 80° C. for 1.5 hour. The reaction mixture was cooled to RT then filtered through a diatomaceous earth pad, then concentrated in vacuo. The resultant residue was subjected to flash chromatography (SiO₂—12 g; gradient elution: 5-30% ethyl acetate/hexane over 30 min at 25 mL/min) to afford 5-bromo-2-cyclopropyl-7-methyl-2,3-dihydro-isoindol-1-one as a white solid upon concentration (179 mg, 54%). ¹H NMR (300 MHz, CDCl₃) δ 7.36 (s, 1H), 7.34 (s, 1H), 4.23 (s, 2H), 2.88 (sept, 1H), 2.68 (s, 3H), 0.802-0.939 (m, 4H); m/z (AP+) M+1=268.1; HPLC t_{R}=2.33 min.

### b) 2-Cyclopropyl-7-methyl-5-pyridin-3-yl-2,3-dihydro-isoindol-1-one

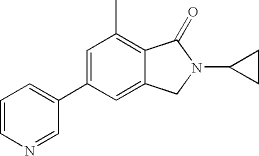

A thick-walled glass vial was charged with a stir bar, 5-bromo-2-cyclopropyl-7-methyl-2,3-dihydro-isoindol-1-one (65 mg, 0.244 mmol), pyridine-3-boronic acid (30 mg, 0.244 mmol), dichlorobis(triphenylphosphine)-palladium (II) (1.7 mg, 0.0024 mmol), Cs₂CO₃ (95 mg, 0.293 mmol) and DME/H₂O/EtOH (7:3:2—1.0 mL). The reaction vial was sealed and subjected to microwave radiation for 160 seconds at 150° C., fixed hold time. The resultant black slurry was extracted with ethyl acetate (3×3 mL), filtered through a magnesium silicate pad, dried over Na₂SO₄, filtered through a plug of cotton and concentrated in vacuo. The resultant residue was subjected to flash chromatography (SiO₂—4 g; gradient elution: 0-50% EtOAc/methylene chloride over 30 min at 15 mL/min) to afford 2-cyclopropyl-7-methyl-5-pyridin-3-yl-2,3-dihydro-isoindol-1-one as a white solid upon concentration (47 mg, 64%). ¹H NMR (300 MHz, CDCl₃) δ 8.85 (s, 1H), 8.65 (d, 1H), 7.91 (dd, 1H), 7.38-7.40 (m, 3H), 4.33 (s, 2H), 2.91-2.98 (m, 1H), 2.79 (s, 3H), 0.866-0.939 (m, 4H); m/z (AP+) M+1=265.3; HPLC t_{R}=1.64 min.

### EXAMPLE 2

### N-(3-(7-Methyl-2-(3-methylbutan-2-yl)-1-oxoisoindolin-5-yl)phenyl)methane-sulfonamide

N-(3-(7-Methyl-2-(3-methylbutan-2-yl)-1-oxoisoindolin-5-yl)phenyl)methane-sulfonamide was prepared as illustrated in Scheme 2.

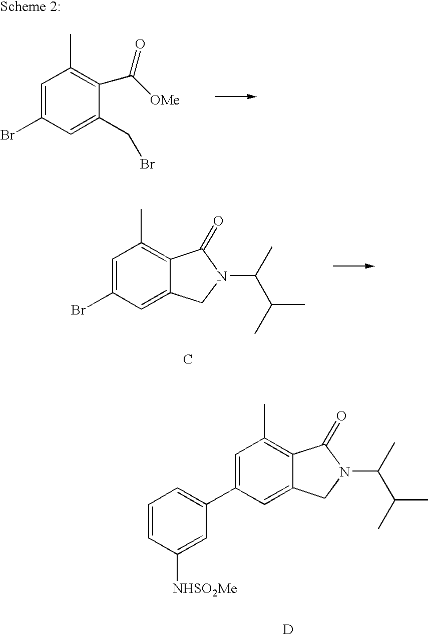

### a) 5-Bromo-2-(1,2-dimethyl-propyl)-7-methyl-2,3-dihydro-isoindol-1-one

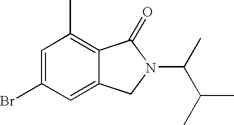

To a stirred solution of 4-bromo-2-bromomethyl-6-methyl-benzoic acid methyl ester (400 mg, 1.24 mmol) in 25 mL of ACN was added 1,2-dimethylpropylamine (156 μL, 1.36 mmol), stirring for about five minutes prior to the addition of K₂CO₃ (343 mg, 2.48 mmol). The reaction vessel was sealed and heated to 80° C. for 16 hours. Upon cooling to RT, the mixture was then filtered through a 0.45 μm GMF filter and concentrated in vacuo. The resultant residue was subjected to flash chromatography (SiO₂—12 g; gradient elution: 1% EtOAc/hexane over 1 min then 1-8.5% EtOAc/hexane over 10 min at 40 mL/min) to yield 5-bromo-2-(1,2-dimethyl-propyl)-7-methyl-2,3-dihydro-isoindol-1-one as a white solid upon concentration (238 mg, 65%). m/z (ES+) M+=296.06; HPLC t_{R}=2.65 min.

### b) N-(3-(7-methyl-2-(3-methylbutan-2-yl)-1-oxoisoindolin-5-yl)phenyl)methanesulfonamide

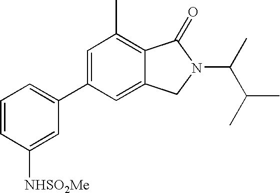

N-(3-(7-methyl-2-(3-methylbutan-2-yl)-1-oxoisoindolin-5-yl)phenyl)-methanesulfonamide was synthesized in an analogous manner as described in Scheme 2, using (3-methyl-sulfonylaminophenyl)boronic acid. The residue was subjected to flash chromatography (SiO₂—4 g; gradient elution: 25% EtOAc/hexane over 1 min then 25-80% EtOAc/hexane over 10 min at 20 mL/min) giving rise to the title compound as a white foam (94 mg, 62%). ¹H NMR (300 MHz, CDCl₃) δ 7.79 (s, 1H), 7.55 (s, 1H), 7.31-7.44 (m, 5H), 4.30 (app dd, J=28.7, 17.3 Hz, 2H), 4.09-4.18 (m, 1H), 3.06 (s, 3H), 2.76 (s, 3H), 1.83 (dquintet, J=9.5, 6.6 Hz, 1H), 1.27 (d, J=6.6 Hz, 3H), 1.01 (d, J=6.6 Hz, 3H), 0.86 (d, J=6.6 Hz, 3H). m/z (ES+) M+1=387.1; HPLC t_{R}=2.20 min.

### EXAMPLE 3

### 2-(2-Ethoxy-1-methylethyl)-7-methyl-5-pyridin-3-ylisoindolin-1-one

2-(2-Ethoxy-1-methylethyl)-7-methyl-5-pyridin-3-ylisoindolin-1-one was prepared as illustrated in Scheme 3.

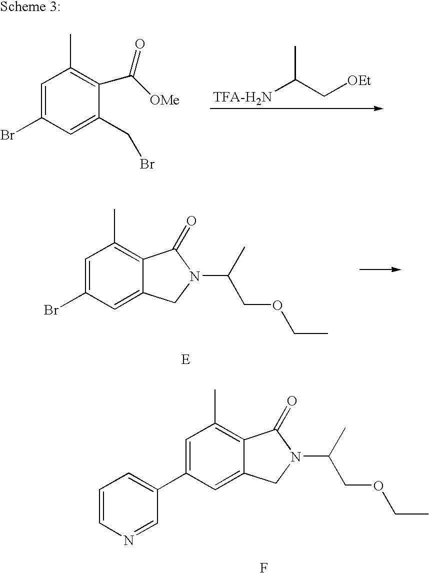

### a) di-tert-butyl (2-ethoxy-1-methylethyl)imidodicarbonate

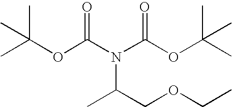

1-Ethoxypropan-2-ol (4.00 g, 38.41 mmol), triphenylphosphine (10.07 g, 38.41 mmol) and di-tert-butyl imidodicarbonate (8.34 g, 38.41 mmol) were combined in 50 mL of anhydrous THF followed by the dropwise addition of diisopropylazodicarboxylate (7.61 mL, 38.41 mmol) over 20 min. The reaction was allowed to stir overnight with warming to room temperature. The volatiles were removed under reduced pressure and the residue filtered through a plug of silica gel which was washed with anhydrous ethyl ether. The volatiles were removed under reduced pressure and the residue purified by flash chromatography (SiO₂ 330 g; elution 0-20% EtOAc/hexane over 1 hour at 100 mL/min) to afford the title compound as a viscous oil (5.28 g, 45%). ¹H NMR (300 MHz, CDCl₃) δ 4.40 (m 1H), 3.69 (m, 1H) 3.42 (m, 3H), 1.46 (s, 18H), 1.22 (d, 3H), 1.12 (t, 3H). m/z (AP+) M+1=304.3; HPLC t_{R}=2.85 min.

### b) (2-ethoxy-1-methylethyl)amine Trifluoroacetic Acid Salt

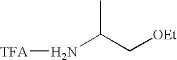

Di-tert-butyl (2-ethoxy-1-methylethyl)imidodicarbonate (5.28 gr, 17.40 mmol) was dissolved in 150 mL of anhydrous DCM followed by the addition of 15 mL of TFA and the reaction stirred at room temperature. After 1 hour, the starting material was consumed and the volatiles were removed under reduced pressure to give the title compound as a viscous TFA salt (3.74 g, 99%). ¹H NMR (300 MHz, DMSO-d₆) δ 7.87 (bs, 3H), 5.20 (m, 1H), 3.49 (m, 2H), 3.36 (m, 2H), 1.16 (m, 6H).

### c) 5-bromo-2-(2-ethoxy-1-methylethyl)-7-methylisoindolin-1-one

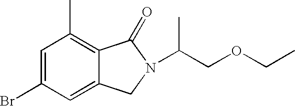

Methyl 4-bromo-2-(bromomethyl)-6-methylbenzoate (0.5 gr, 1.55 mmol), potassium carbonate (1.28 g, 9.31 mmol), boric acid (0.096 g, 1.55 mmol) and (2-ethoxy-1-methylethyl)amine trifluoroacetic acid salt (0.674 g, 3.10 mmol) were combined in 100 mL of anhydrous ACN and heated to reflux overnight. The solution was filtered through a pad of silica gel and rinsed with EtOAc. The volatiles were removed under reduced pressure and the residue purified by flash chromatography (SiO₂ 40 g; elution 0-20% ethyl acetate/hexane over 30 min at 40 mL/min). The title product was isolated as an oil that gradually solidified (0.350 g, 72%). ¹H NMR (300 MHz, CDCl₃) δ 7.41 (s, 1H), 7.35 (s, 1H), 4.63 (m, 1H), 4.38 (dd, 2H), 3.59-3.45 (m, 4H), 2.72 (s, 3H), 1.32 (d, 3H), 1.17 (t, 3H). m/z (AP+) M+1=313.1; HPLC t_{R}=2.41 min.

### d) 2-(2-ethoxy-1-methylethyl)-7-methyl-5-pyridin-3-ylisoindolin-1-one

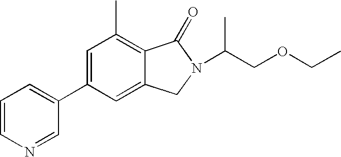

5-Bromo-2-(2-ethoxy-1-methylethyl)-7-methylisoindolin-1-one (0.175 g, 0.56 mmol), pyridin-3-ylboronic acid (0.103 g, 0.84 mmol), cesium carbonate (0.219 g, 0.672 mmol) and bis(triphenylphosphine)palladium dichloride (0.004 g, 0.0056 mmol) were dissolved in DME/H₂O/EtOH (7:3:2-4 mL), the reaction vial was sealed and subjected to microwave irradiation for 5 minutes at 150° C., fixed hold time. The reaction mixture was placed in a separatory funnel, diluted with 200 mL of EtOAc and washed twice with 50 mL of brine. The organics were dried over Na₂SO₄, filtered and the volatiles removed under reduced pressure. The organics were purified by flash chromatography (SiO₂ 12 g; elution 0-65% EtOAc/hexane over 30 min at 25 mL/min). The title product was isolated as of a semi-solid material (147.2 mg, 84%). ¹H NMR (300 MHz, CDCl₃) δ 8.87 (s, 1H), 8.64 (m, 1H), 7.89 (m, 1H), 7.45 (m, 1H), 7.38 (m, 2H), 4.68 (m, 1H), 4.47 (dd, 2H), 3.64-3.45 (m, 4H), 2.82 (s, 3H), 1.34 (d, 3H), 1.18 (t, 3H). m/z (AP+) M+1=311.2; HPLC t_{R}=1.69 min.

Compounds of Examples 5 through 55 illustrated in Table 1, were synthesized using methods analogous to those described herein.

TABLE 1

| | | | | | | LC |
|---|---|---|---|---|---|---|
| Ex. | | Preparation | Purification | m/z M + 1 | MS | tR |
| No. | Structure | Scheme | Method | (ionization) | Type | (min) |
| 5 | 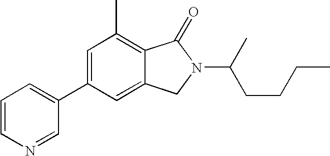 | 1 | A | 309.4 | AP+ | 2.35 |
| 6 | 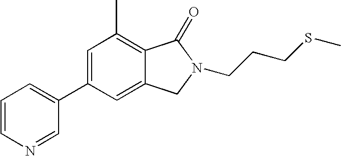 | 1 | A | 313.3 | AP+ | 1.89 |
| 7 | 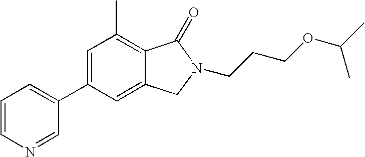 | 1 | A | 325.4 | AP+ | 1.9 |
| 8 | 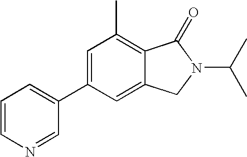 | 1 | A | 267.3 | AP+ | 1.74 |
| 9 | 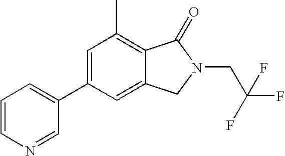 | 1 | A | 307.3 | AP+ | 1.9 |
| 10 | 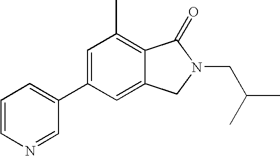 | 1 | A | 281.2 | AP+ | 1.93 |
| 11 | 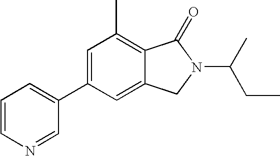 | 1 | A | 281.3 | AP+ | 1.89 |
| 12 | 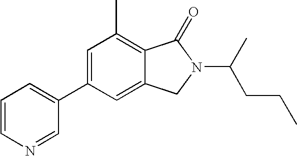 | 1 | A | 295.3 | AP+ | 2.12 |
| 13 | 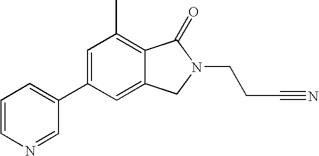 | 1 | A | 278.2 | AP+ | 1.48 |
| 14 | 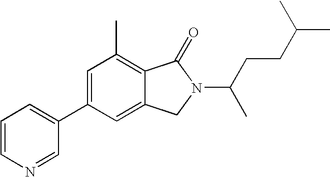 | 1 | A | 323.3 | AP+ | 2.52 |
| 15 | 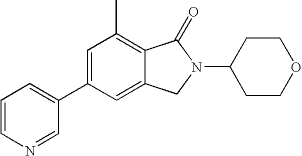 | 1 | A | 309.3 | AP+ | 1.65 |
| 16 | 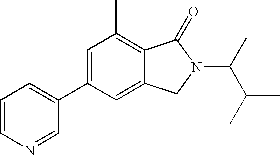 | 3 | A | 295.2 | ES+ | 2.06 |
| 17 | 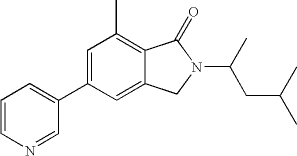 | 3 | A | 309.2 | ES+ | 2.31 |
| 18 | 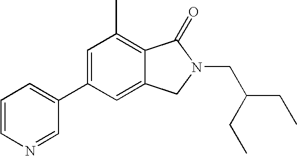 | 3 | A, B | 309.2 | ES+ | 2.34 |
| 19 | 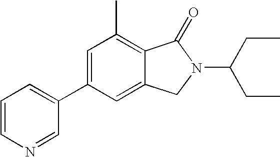 | 3 | A | 295.2 | ES+ | 2.04 |
| 20 | 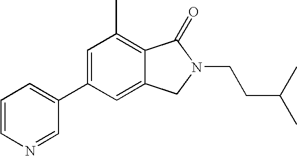 | 3 | A | 295.2 | ES+ | 2.16 |
| 21 | 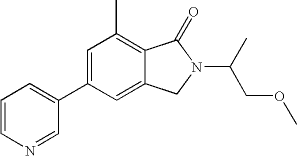 | 1 | A | 297.4 | AP+ | 1.68 |
| 22 | 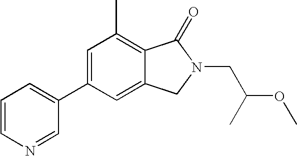 | 1 | A | 297.4 | AP+ | 1.68 |
| 23 | 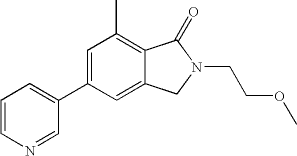 | 1 | A | 283.3 | AP+ | 1.57 |
| 24 | 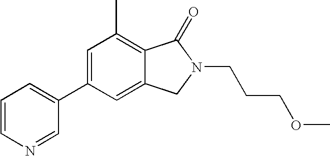 | 1 | A | 297.4 | AP+ | 1.65 |
| 25 | 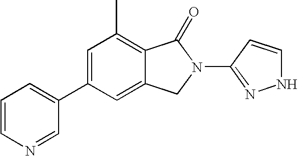 | 1 | A | 291.1 | AP+ | 1.56 |
| 26 | 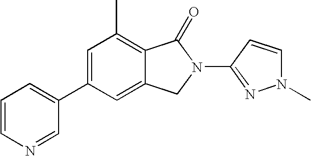 | 1 | A | 305.3 | AP+ | 1.78 |
| 27 | 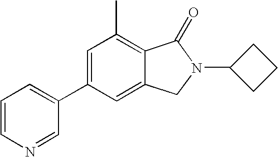 | 3 | A | 279.1 | ES+ | 1.86 |
| 28 | 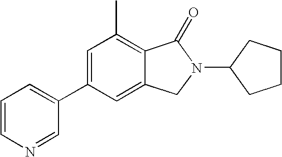 | 3 | A | 293.2 | ES+ | 2.01 |
| 29 | 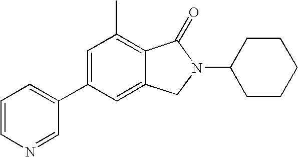 | 3 | A | 307.2 | ES+ | 2.17 |
| 30 | 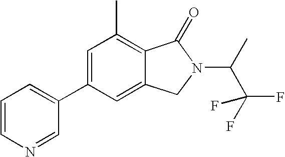 | 1 | A | 321.1 | AP+ | 2.01 |
| 31 | 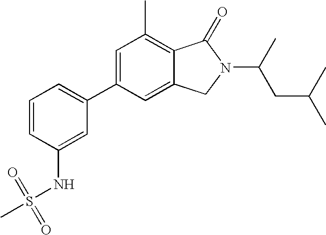 | 3 | A | 401.2 | ES+ | 2.62 |
| 32 | 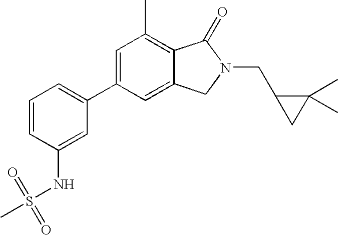 | 2 | A | 399.2 | ES+ | 2.58 |
| 33 | 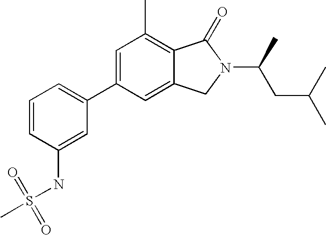 | 3 | A | 401.2 | ES+ | 2.62 |
| 34 | 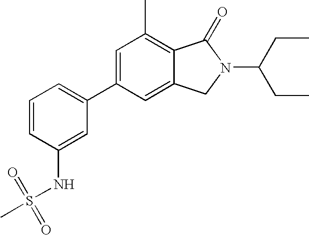 | 3 | A | 387.1 | ES+ | 2.20 |
| 35 | 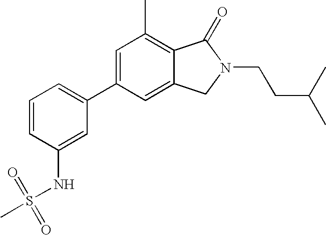 | 3 | A | 387.1 | ES+ | 2.28 |
| 36 | 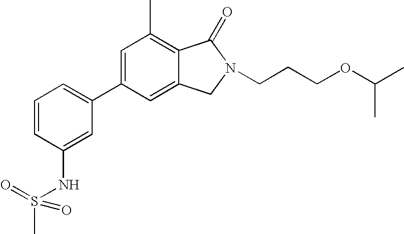 | 3 | A | 417.4 | AP+ | 2.39 |
| 37 | 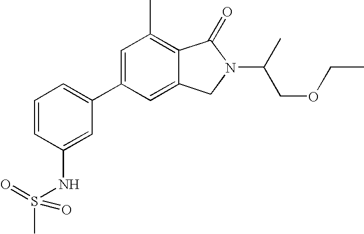 | 3 | A | 403.4 | AP+ | 2.36 |
| 38 | 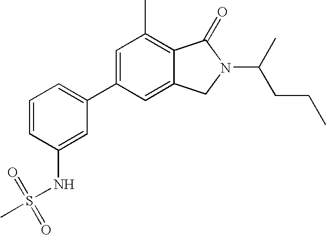 | 3 | A | 387.4 | AP+ | 2.56 |
| 39 | 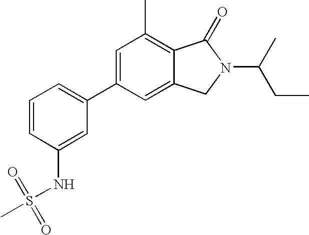 | 3 | A | 373.1 | AP+ | 2.35 |
| 40 | 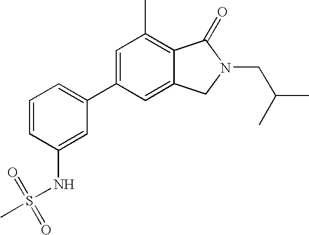 | 3 | A | 373.4 | AP+ | 2.45 |
| 41 | 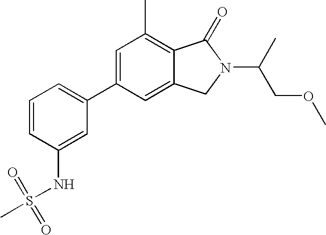 | 3 | A | 389.3 | AP+ | 2.24 |
| 42 | 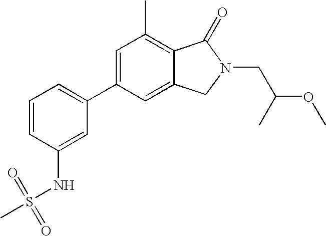 | 3 | A | 389.1 | AP+ | 2.15 |
| 43 | 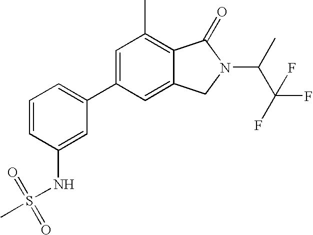 | 3 | A | 413.1 | AP+ | 2.45 |
| 44 | 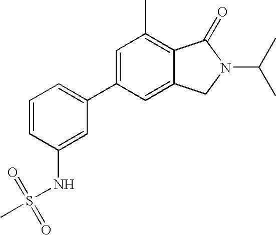 | 3 | A | 359.4 | AP+ | 2.31 |
| 45 | 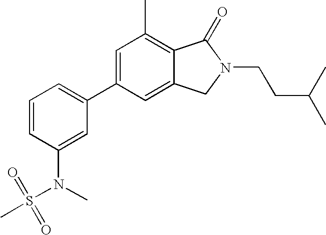 | NH by scheme 3, then NMe by std chemistry | A | 401.1 | ES+ | 2.34 |
| 46 | 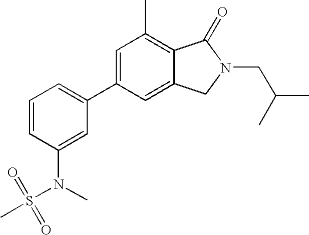 | NH by scheme 3, then NMe by std chemistry | A | 387.4 | AP+ | 2.60 |
| 47 | 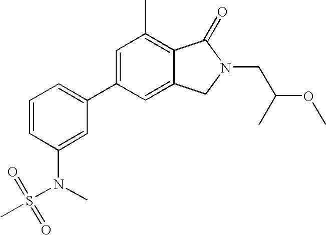 | NH by scheme 3, then NMe by std chemistry | A | 403.1 | AP+ | 2.29 |
| 48 | 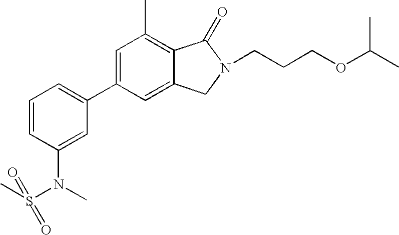 | NH by scheme 3, then NMe by std chemistry | A | 431.4 | AP+ | 2.54 |
| 49 | 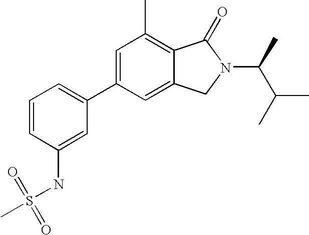 | 3 | A | 387.1 | ES+ | 2.21 |
| 50 | 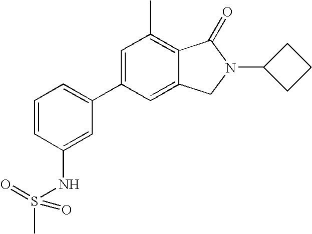 | 3 | A | 371.1 | AP+ | 2.39 |
| 51 | 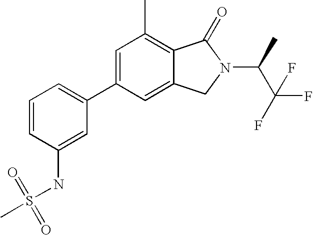 | 3 | A | 413.1 | AP+ | 2.48 |
| 52 | 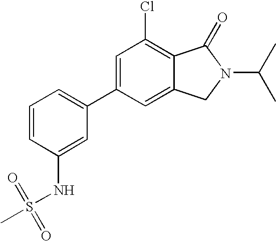 | Scheme 3 using 4- bromo-2- bromomethyl- 6-chloro- benzoic acid methyl ester isoindolone precursor | A | 379.1 | AP+ | 2.26 |
| 53 | 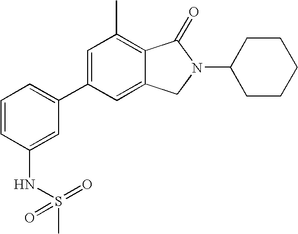 | 3 | A | 399.2 | AP+ | 2.59 |
| 54 | 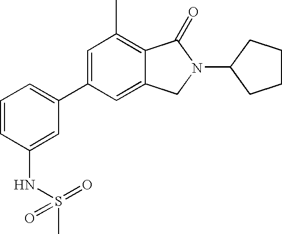 | 3 | A | 385.1 | AP+ | 2.48 |
| 55 | 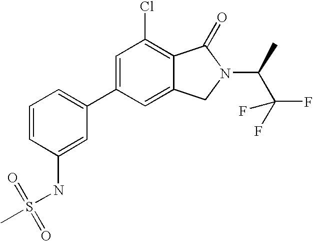 | Scheme 3 using 4- bromo-2- bromomethyl- 6-chloro- benzoic acid methyl ester isoindolone precursor | A | 433.0 | AP+ | 2.46 |

Exemplary compounds illustrated in Table 1 are:
Example 4 2-(Hexan-2-yl)-7-methyl-5-(pyridin-3-yl)isoindolin-1-one;
Example 5 7-Methyl-2-(3-(methylthio)propyl)-5-(pyridin-3-yl)isoindolin-1-one;
Example 6 2-(3-Isopropoxypropyl)-7-methyl-5-(pyridin-3-yl)isoindolin-1-one;
Example 7 2-Isopropyl-7-methyl-5-(pyridin-3-yl)isoindolin-1-one;
Example 8 7-Methyl-5-(pyridin-3-yl)-2-(2,2,2-trifluoroethyl)isoindolin-1-one;
Example 9 2-Isobutyl-7-methyl-5-(pyridin-3-yl)isoindolin-1-one;
Example 10 2-sec-Butyl-7-methyl-5-(pyridin-3-yl)isoindolin-1-one;
Example 11 7-Methyl-2-(pentan-2-yl)-5-(pyridin-3-yl)isoindolin-1-one;
Example 12 3-(7-Methyl-1-oxo-5-(pyridin-3-yl)isoindolin-2-yl)propanenitrile;
Example 13 7-Methyl-2-(5-methylhexan-2-yl)-5-(pyridin-3-yl)isoindolin-1-one;
Example 14 7-Methyl-5-(pyridin-3-yl)-2-(tetrahydro-2H-pyran-4-yl)isoindolin-1-one;
Example 15 7-Methyl-2-(3-methylbutan-2-yl)-5-(pyridin-3-yl)isoindolin-1-one 2-hydroxypropane-1,2,3-tricarboxylate;
Example 16 7-Methyl-2-(4-methylpentan-2-yl)-5-(pyridin-3-yl)isoindolin-1-one 2-hydroxypropane-1,2,3-tricarboxylate;
Example 17 2-(2-Ethylbutyl)-7-methyl-5-(pyridin-3-yl)isoindolin-1-one 2-hydroxypropane-1,2,3-tricarboxylate;
Example 18 7-Methyl-2-(pentan-3-yl)-5-(pyridin-3-yl)isoindolin-1-one 2-hydroxypropane-1,2,3-tricarboxylate;
Example 19 2-Isopentyl-7-methyl-5-(pyridin-3-yl)isoindolin-1-one 2-hydroxypropane-1,2,3-tricarboxylate;
Example 20 2-(1-Methoxypropan-2-yl)-7-methyl-5-(pyridin-3-yl)isoindolin-1-one;
Example 21 2-(2-Methoxypropyl)-7-methyl-5-(pyridin-3-yl)isoindolin-1-one;
Example 22 2-(2-Methoxyethyl)-7-methyl-5-(pyridin-3-yl)isoindolin-1-one;
Example 23 2-(3-Methoxypropyl)-7-methyl-5-(pyridin-3-yl)isoindolin-1-one;
Example 24 7-Methyl-2-(1H-pyrazol-3-yl)-5-(pyridin-3-yl)isoindolin-1-one;
Example 25 7-Methyl-2-(1-methyl-1H-pyrazol-3-yl)-5-(pyridin-3-yl)isoindolin-1-one;
Example 26 2-Cyclobutyl-7-methyl-5-(pyridin-3-yl)isoindolin-1-one 2-hydroxypropane-1,2,3-tricarboxylate;
Example 27 2-Cyclopentyl-7-methyl-5-(pyridin-3-yl)isoindolin-1-one 2-hydroxypropane-1,2,3-tricarboxylate;
Example 28 2-Cyclohexyl-7-methyl-5-(pyridin-3-yl)isoindolin-1-one 2-hydroxypropane-1,2,3-tricarboxylate;
Example 29 7-Methyl-5-(pyridin-3-yl)-2-(1,1,1-trifluoropropan-2-yl)isoindolin-1-one;
Example 30 N-(3-(7-Methyl-2-(4-methylpentan-2-yl)-1-oxoisoindolin-5-yl)phenyl)methanesulfonamide;
Example 31 N-(3-(7-Methyl-2-(4-methylpentan-2-yl)-1-oxoisoindolin-5-yl)phenyl)methanesulfonamide;
Example 32 N-(3-(2-((2,2-Dimethylcyclopropyl)methyl)-7-methyl-1-oxoisoindolin-5-yl)phenyl)methanesulfonamide;
Example 33 N-{3-[2-((S)-1,3-Dimethyl-butyl)-7-methyl-1-oxo-2,3-dihydro-1H-isoindol-5-yl]-phenyl}-methanesulfonamide;
Example 34 N-{3-[2-(1-Ethyl-propyl)-7-methyl-1-oxo-2,3-dihydro-1H-isoindol-5-yl]-phenyl}-methanesulfonamide;
Example 35 N-{3-[7-Methyl-2-(3-methyl-butyl)-1-oxo-2,3-dihydro-1H-isoindol-5-yl]-phenyl}-methanesulfonamide;
Example 36 N-{3-[2-(3-Isopropoxy-propyl)-7-methyl-1-oxo-2,3-dihydro-1H-isoindol-5-yl]-phenyl}-methanesulfonamide;
Example 37 N-{3-[2-(2-Ethoxy-1-methyl-ethyl)-7-methyl-1-oxo-2,3-dihydro-1H-isoindol-5-yl]-phenyl}-methanesulfonamide;
Example 38 N-{3-[7-Methyl-2-(1-methyl-butyl)-1-oxo-2,3-dihydro-1H-isoindol-5-yl]-phenyl}-methanesulfonamide;
Example 39 N-{3-(2-sec-Butyl-7-methyl-1-oxo-2,3-dihydro-1H-isoindol-5-yl]-phenyl}-methanesulfonamide;
Example 40 N-[3-(2-Isobutyl-7-methyl-1-oxo-2,3-dihydro-1H-isoindol-5-yl)-phenyl]-methanesulfonamide;
Example 41 N-{3-[2-(2-Methoxy-1-methyl-ethyl)-7-methyl-1-oxo-2,3-dihydro-1H-isoindol-5-yl]-phenyl}-methanesulfonamide;
Example 42 N-{3-[2-(2-Methoxy-propyl)-7-methyl-1-oxo-2,3-dihydro-1H-isoindol-5-yl]-phenyl}-methanesulfonamide;
Example 43 N-{3-[7-Methyl-1-oxo-2-(2,2,2-trifluoro-1-methyl-ethyl)-2,3-dihydro-1H-isoindol-5-yl]-phenyl}-methanesulfonamide;
Example 44 N-[3-(2-Isopropyl-7-methyl-1-oxo-2,3-dihydro-1H-isoindol-5-yl)-phenyl]-methanesulfonamide;
Example 45 N-Methyl-N-{3-[7-methyl-2-(3-methyl-butyl)-1-oxo-2,3-dihydro-1H-isoindol-5-yl]-phenyl}-methanesulfonamide;
Example 46 N-[3-(2-Isobutyl-7-methyl-1-oxo-2,3-dihydro-1H-isoindol-5-yl)-phenyl]-N-methyl-methanesulfonamide;
Example 47 N-{3-[2-(2-Methoxy-propyl)-7-methyl-1-oxo-2,3-dihydro-1H-isoindol-5-yl]-phenyl}-N-methyl-methanesulfonamide;
Example 48 N-{3-[2-(3-Isopropoxy-propyl)-7-methyl-1-oxo-2,3-dihydro-1H-isoindol-5-yl]-phenyl}-N-methyl-methanesulfonamide;
Example 49 N-{3-[2-((S)-1,2-Dimethyl-propyl)-7-methyl-1-oxo-2,3-dihydro-1H-isoindol-5-yl]-phenyl}-methanesulfonamide;
Example 50 N-[3-(2-Cyclobutyl-7-methyl-1-oxo-2,3-dihydro-1H-isoindol-5-yl)-phenyl]-methanesulfonamide;
Example 51 N-{3-[7-Methyl-1-oxo-2-((S)-2,2,2-trifluoro-1-methyl-ethyl)-2,3-dihydro-1H-isoindol-5-yl]-phenyl}-methanesulfonamide;
Example 52 N-[3-(7-Chloro-2-isopropyl-1-oxo-2,3-dihydro-1H-isoindol-5-yl)-phenyl]-methanesulfonamide;
Example 53 N-[3-(2-Cyclohexyl-7-methyl-1-oxo-2,3-dihydro-1H-isoindol-5-yl)-phenyl]-methanesulfonamide;
Example 54 N-[3-(2-Cyclopentyl-7-methyl-1-oxo-2,3-dihydro-1H-isoindol-5-yl)-phenyl]-methanesulfonamide, and
Example 55 N-{3-[7-Chloro-1-oxo-2-((S)-2,2,2-trifluoro-1-methyl-ethyl)-2,3-dihydro-1H-isoindol-5-yl]-phenyl}-methanesulfonamide.

The pharmacological properties of compounds of Formula I can be assessed using standard assays for functional activity. Examples of glutamate receptor assays are well known in the art as described in, for example, Aramori et al., 1992, Neuron, 8:757; Tanabe et al., 1992, Neuron, 8:169; Miller et al., 1995, J. Neuroscience, 15:6103; Balazs, et al., 1997, J. Neurochemistry, 69:151. Conveniently, compounds can be studied by means of an assay that measures the mobilization of intracellular calcium, [Ca²⁺]ᵢ in cells expressing mGluR₂. Generally, described compounds are active in assays described herein at concentrations (or with EC₅₀ values) less than 10 μM.

Fluorometric Imaging Plate Reader (FLIPR) analysis can be used to detect allosteric activators of mGluR2 via calcium mobilization. A clonal HEK 293 cell line expressing a chimeric mGluR2/CaR construct comprising the extracellular and transmembrane domains of human mGluR2 and the intracellular domain of the human calcium receptor, fused to the promiscuous chimeric protein G_{αqi5} was used. Activation of this construct by agonists or allosteric activators resulted in stimulation of the PLC pathway and the subsequent mobilization of intracellular Ca²⁺ which was measured via FLIPR analysis. At 24-hours prior to analysis, the cells were trypsinized and plated in DMEM at 100,000 cells/well in black sided, clear-bottom, collagen I coated, 96-well plates. The plates were incubated under 5% CO₂ at 37° C. overnight. Cells were loaded with 6 μM fluo-3 acetoxymethylester (Molecular Probes, Eugene Oreg.) for 60 minutes at room temperature. All assays were performed in a buffer containing 126 mM NaCl, 5 mM KCl, 1 mM MgCl₂, 1 mM CaCl₂, 20 mM Hepes, 0.06 μM DCG-IV (a Group II mGluR selective agonist), supplemented with 1.0 mg/ml D-glucose and 1.0 mg/ml BSA fraction IV (pH 7.4).

FLIPR experiments were done using a laser setting of 0.8 W and a 0.4 second CCD camera shutter speed. Extracellular fluo-3 was washed off and cells were maintained in 160 μL of buffer and placed in the FLIPR. An addition of test compound (0.01 μM to 30 μM in duplicate) was made after 10 seconds of baseline fluorescent readings were recorded on FLIPR. Fluorescent signals were then recorded for an additional 75 seconds at which point a second addition of DCG-IV (0.2 μM) was made and fluorescent signals were recorded for an additional 65 seconds. Fluorescent signals were measured as the peak height of the response within the sample period. Data was analyzed using Assay Explorer, and EC₅₀ and Eₘₐₓ values (relative to maximum DCG-IV effect) were calculated using a four parameter logistic equation.

A [³⁵S]-GTPγS binding assay was used to functionally assay mGluR2 receptor activation. The allosteric activator activity of compounds at the human mGluR2 receptor were measured using a [³⁵S]-GTPγS binding assay with membranes prepared from CHO cells which stably express the human mGluR2. The assay is based upon the principle that agonists bind to G-protein coupled receptors to stimulate GDP-GTP exchange at the G-protein. Since [³⁵S]-GTPγS is a non-hydrolyzable GTP analog, it can be used to provide an index of GDP-GTP exchange and, thus, receptor activation. The GTPγS binding assay therefore provides a quantitative measure of receptor activation.

Membranes were prepared from CHO cells stably transfected with human mGluR2. Membranes (30 μg protein) were incubated with test compound (3 nM to 300 μM) for 15 minutes at room temperature prior to the addition of 1 μM glutamate, and incubated for 30 min at 30° C. in 500 μL assay buffer (20 mM HEPES, 100 mM NaCl, 10 mM MgCl₂), containing 30 μM GDP and 0.1 nM [³⁵S]-GTPγS (1250 Ci/mmol). Reactions were carried out in triplicate in 2 mL polypropylene 96-well plates. Reactions were terminated by vacuum filtration using a Packard 96-well harvester and Unifilter-96, GF/B filter microplates. The filter plates were washed 4×1.5 mL with ice-cold wash buffer (10 mM sodium phosphate buffer, pH 7.4). The filter plates were dried and 35 μL of scintillation fluid (Microscint 20) was added to each well. The amount of radioactivity bound was determined by counting plates on the Packard TopCount. Data was analyzed using GraphPad Prism, and EC₅₀ and Eₘₐₓ values (relative to the maximum glutamate effect) were calculated using non-linear regression.

1. A compound in accord with Formula I, 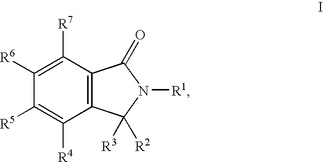
wherein:
R¹ is —CHR⁸R⁹;
R², R³ and R⁴ are H;
R⁶ and R⁷ are independently selected from the group consisting of H, halogen and C₁₋₆-alkyl;
R⁵ is selected from the group consisting of C₁₋₆-alkyl, C₀₋₆-alkylaryl, C₀₋₆-alkylheteroaryl and C₀₋₆-alkylheterocyclyl; wherein, when chemically-feasible, R⁵ may be substituted by one or more A, and wherein any cyclic moiety is optionally fused to a 5- to 7-membered ring that may have one or more heteroatoms independently selected from the group consisting of N, O and S;
A is selected from the group consisting of C₁₋₆-alkyl, C₀₋₆-alkylaryl, C₀₋₆-alkylheteroaryl, C₀₋₆-alkylheterocyclyl, C₀₋₆-alkyl(CO)N(R¹⁰)₂, C₀₋₆-alkylNR¹⁰(CO)R¹⁰, C₀₋₆-alkyl(SO₂)N(R¹⁰)₂, C₀₋₆-alkylNR¹⁰(SO₂)R¹⁰ and a 5- to 7-membered ring that may have one or more heteroatoms independently selected from the group consisting of N, O and S, wherein said 5- to 7-membered ring is optionally substituted by one or more R¹⁰;
R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;
n is 1, 2, 3, 4, 5 or 6;
X is S or O, and
R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;
or a pharmaceutically acceptable salt, hydrate, solvate, optical isomer, or combination thereof.

2. A compound according to claim 1, wherein:
R¹ is —CHR⁸R⁹;
R², R³ and R⁴ are H;
R⁶ is selected from the group consisting of H, halogen and C₁₋₆-alkyl;
R⁷ is selected from the group consisting of halogen and C₁₋₆-alkyl;
R⁵ is a 5- to 7-membered ring that may have one or more heteroatoms independently selected from the group consisting of N, O and S, wherein, when chemically-feasible, R⁵ may be substituted by one or more A;
A is selected from the group consisting of C₁₋₆-alkyl, C₀₋₆-alkylaryl, C₀₋₆-alkylheteroaryl, C₀₋₆-alkylheterocyclyl, C₀₋₆-alkyl(CO)N(R¹⁰)₂, C₀₋₆-alkylNR¹⁰(CO)R¹⁰, C₀₋₆-alkyl(SO₂)N(R¹⁰)₂, C₀₋₆-alkylNR¹⁰(SO₂)R¹⁰ and a 5- to 7-membered ring that may have one or more heteroatoms independently selected from the group consisting of N, O and S, wherein said 5- to 7-membered ring is optionally substituted by one or more R¹⁰;
R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;
n is 1, 2 or 3;
X is S or O;
R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;
or a pharmaceutically acceptable salt, hydrate, solvate, optical isomer, or combination thereof.

3. A compound according to claim 1, wherein:
R¹ is —CHR⁸R⁹;
R², R³ and R⁴ are H;
R⁶ is selected from the group consisting of H, halogen and C₁₋₆-alkyl;
R⁷ is selected from the group consisting of halogen and C₁₋₆-alkyl;
R⁵ is a 5- to 7-membered ring that may have one or more heteroatoms independently selected from the group consisting of N, O and S, wherein, where chemically-feasible, R⁵ may be substituted by one or more A, and wherein any cyclic moiety is optionally fused to a 5- to 7-membered ring that may have one or more heteroatoms independently selected from the group consisting of N, O and S;
A is C₀₋₆-alkyl(CO)N(R¹⁰)₂, C₀₋₆-alkylNR¹⁰(CO)R¹⁰, C₀₋₆-alkyl(SO₂)N(R¹⁰)₂ or C₀₋₆-alkylNR¹⁰(SO₂)R¹⁰;
R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;
n is 1, 2 or 3;
X is S or O, and
R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;
or a pharmaceutically acceptable salt, hydrate, solvate, optical isomer, or combination thereof.

4. A compound according to claim 1, wherein:
R¹ is —CHR⁸R⁹;
R², R³ and R⁴ are H;
R⁶ is selected from the group consisting of H, halogen and C₁₋₆-alkyl;
R⁷ is selected from the group consisting of halogen and C₁₋₆-alkyl;
R⁵ is phenyl or pyridyl;
A is C₀₋₆-alkyl(CO)N(R¹⁰)₂, C₀₋₆-alkylNR¹⁰(CO)R¹⁰, C₀₋₆-alkyl(SO₂)N(R¹⁰)₂ or C₀₋₆-alkylNR¹⁰(SO₂)R¹⁰;
R⁸ and R⁹ are independently selected from H, C₁₋₆alkyl, C₁₋₆alkoxyC₁₋₆alkyl-, —(CH₂)ₙ—X—R¹⁰, C₁₋₆-fluoroalkyl, C₁₋₆-perfluoroalkyl or CN, or R⁸ and R⁹ in combination a form a C₃₋₇-cycloalkyl group or a heterocyclyl group with the proviso that R⁸ and R⁹ are not both H;
n is 1, 2 or 3;
X is S or O, and
R¹⁰ at each occurrence is independently selected from the group consisting of H, C₁₋₆-alkyl, C₀₋₆-alkylaryl, C₀₋₆-alkylheteroaryl and C₀₋₆-alkylheterocyclyl wherein any cyclic moiety is optionally fused to a 5- to 7-membered ring that may have one or more heteroatoms independently selected from the group consisting of N, O and S and any cyclic moiety is optionally substituted with a substituent selected from halo, hydroxyl, alkyl, alkoxy, haloalkyl and haloalkoxy;
or a pharmaceutically acceptable salt, hydrate, solvate, optical isomer, or combination thereof.

5. A compound selected from:
2-Cyclopropyl-7-methyl-5-pyridin-3-yl-2,3-dihydro-isoindol-1-one;
N-(3-(7-Methyl-2-(3-methylbutan-2-yl)-1-oxoisoindolin-5-yl)phenyl)methane-sulfonamide;
2-(2-Ethoxy-1-methylethyl)-7-methyl-5-pyridin-3-ylisoindolin-1-one;
2-(Hexan-2-yl)-7-methyl-5-(pyridin-3-yl)isoindolin-1-one;
7-Methyl-2-(3-(methylthio)propyl)-5-(pyridin-3-yl)isoindolin-1-one;
2-(3-Isopropoxypropyl)-7-methyl-5-(pyridin-3-yl)isoindolin-1-one;
2-Isopropyl-7-methyl-5-(pyridin-3-yl)isoindolin-1-one;
7-Methyl-5-(pyridin-3-yl)-2-(2,2,2-trifluoroethyl)isoindolin-1-one;
2-Isobutyl-7-methyl-5-(pyridin-3-yl)isoindolin-1-one;
2-sec-Butyl-7-methyl-5-(pyridin-3-yl)isoindolin-1-one;
7-Methyl-2-(pentan-2-yl)-5-(pyridin-3-yl)isoindolin-1-one;
3-(7-Methyl-1-oxo-5-(pyridin-3-yl)isoindolin-2-yl)propanenitrile;
7-Methyl-2-(5-methylhexan-2-yl)-5-(pyridin-3-yl)isoindolin-1-one;
7-Methyl-5-(pyridin-3-yl)-2-(tetrahydro-2H-pyran-4-yl)isoindolin-1-one;
7-Methyl-2-(3-methylbutan-2-yl)-5-(pyridin-3-yl)isoindolin-1-one 2-hydroxypropane-1,2,3-tricarboxylate;
7-Methyl-2-(4-methylpentan-2-yl)-5-(pyridin-3-yl)isoindolin-1-one 2-hydroxypropane-1,2,3-tricarboxylate;
2-(2-Ethylbutyl)-7-methyl-5-(pyridin-3-yl)isoindolin-1-one 2-hydroxypropane-1,2,3-tricarboxylate;
7-Methyl-2-(pentan-3-yl)-5-(pyridin-3-yl)isoindolin-1-one 2-hydroxypropane-1,2,3-tricarboxylate;
2-Isopentyl-7-methyl-5-(pyridin-3-yl)isoindolin-1-one 2-hydroxypropane-1,2,3-tricarboxylate;
2-(1-Methoxypropan-2-yl)-7-methyl-5-(pyridin-3-yl)isoindolin-1-one;
2-(2-Methoxypropyl)-7-methyl-5-(pyridin-3-yl)isoindolin-1-one;
2-(2-Methoxyethyl)-7-methyl-5-(pyridin-3-yl)isoindolin-1-one;
2-(3-Methoxypropyl)-7-methyl-5-(pyridin-3-yl)isoindolin-1-one;
7-Methyl-2-(1H-pyrazol-3-yl)-5-(pyridin-3-yl)isoindolin-1-one;
7-Methyl-2-(1-methyl-1H-pyrazol-3-yl)-5-(pyridin-3-yl)isoindolin-1-one;
2-Cyclobutyl-7-methyl-5-(pyridin-3-yl)isoindolin-1-one 2-hydroxypropane-1,2,3-tricarboxylate;
2-Cyclopentyl-7-methyl-5-(pyridin-3-yl)isoindolin-1-one 2-hydroxypropane-1,2,3-tricarboxylate;
2-Cyclohexyl-7-methyl-5-(pyridin-3-yl)isoindolin-1-one 2-hydroxypropane-1,2,3-tricarboxylate;
7-Methyl-5-(pyridin-3-yl)-2-(1,1,1-trifluoropropan-2-yl)isoindolin-1-one, and
N-(3-(7-Methyl-2-(4-methylpentan-2-yl)-1-oxoisoindolin-5-yl)phenyl)methanesulfonamide.

6. A pharmaceutical composition comprising a compound according to claim 5 and a pharmaceutically acceptable carrier or excipient.

7-9. (canceled)

10. A method for the treatment or prevention of a neurological or psychiatric disorder associated with glutamate dysfunction in an animal in need of such treatment, comprising the step of administering to said animal a therapeutically effective amount of a compound according to claim 5.

11. (canceled)

12. The method according to claim 10, wherein the neurological or psychiatric disorder is selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, and post-traumatic pain, tardive dyskinesia, sleep disorders, narcolepsy, attention deficit/hyperactivity disorder, and conduct disorder.

13. The method according to claim 12, wherein the neurological or psychiatric disorder is selected from Alzheimer's disease, cerebral deficits secondary to prolonged status epilepticus, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, and bipolar disorders.

14. A pharmaceutical composition comprising a compound according to claim 1 and a pharmaceutically acceptable carrier or excipient.

15. A method for the treatment or prevention of a neurological or psychiatric disorder associated with glutamate dysfunction in an animal in need of such treatment, comprising the step of administering to said animal a therapeutically effective amount of a compound according to claim 1.

16. The method according to claim 15, wherein the neurological or psychiatric disorder is selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, and post-traumatic pain, tardive dyskinesia, sleep disorders, narcolepsy, attention deficit/hyperactivity disorder, and conduct disorder.

17. The method according to claim 16, wherein the neurological or psychiatric disorder is selected from Alzheimer's disease, cerebral deficits secondary to prolonged status epilepticus, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, and bipolar disorders.
